SEQUENCE LISTING (1 sequence)

SEQ ID NO: 1 - protein, 315 aa, Microbulbifer thermotolerans
MEQKMRQLDPKLAEWLAGVNAQMAQLKASGFEPTPITARESLANLTRTFVEPGPEMAVCETLVQGGHYQVPVRIYRPQNIEPGPAIIYCHGGGHMAGSVTVYDPICRRVAEHCRRTVISVDYRLAPENPYPAGLQDLLSVVKHLGPALEKKSIDHDGRWILMGDSGGGALCASASRLLQHQHHSISAQVLIYPSLDYTMQTPAIEENGSGYLLEKEKIAWYFDNYFQGTENRREASPLHGEFTKNLPKTLMITAEFCPLRDEGYRYREKLSEAGVACESLHFDDMIHAFLNLEALVPQACEKFYRQVGEFVWGIG

(19) United States
(12) Patent Application Publication
(10) Pub. No.: US 2020/0172884 A1
(43) Pub. Date: Jun. 4, 2020

(54) MICROBULBIFER THERMOTOLERANS LIPASE AND USE THEREOF

(71) Applicant: Henkel AG & Co. KGaA, Duesseldorf (DE)

(72) Inventors: DANIELA HERBST, Duesseldorf (DE); NINA MUSSMANN, Willich (DE); SUSANNE WIELAND, Zons/Dormagen (DE); CLAUDIA LINDNER, Solingen (DE); PHILIP BUSCH, Hamburg (DE); GARABED ANTRANIKIAN, Seevetal-Hittfeld (DE)

(73) Assignee: Henkel AG & Co. KGaA, Duesseldorf (DE)

(21) Appl. No.: 16/621,631

(22) PCT Filed: Jun. 6, 2018
(86) PCT No.: PCT/EP2018/064852

§ 371 Date: Dec. 11, 2019

(30) Foreign Application Priority Data
Jun. 12, 2017 (DE) 10 2017 209 869.4

(51) Int. Cl. C12N 9/20; C11D 3/386

(52) U.S. Cl. CPC C12N 9/20; C12Y 301/01003; C11D 3/38627

(57) ABSTRACT

The present disclosure concerns the field of enzyme technology, in particular the lipolytic action of enzymes on fats and oils as used in detergents or cleaning agents, for example. The present disclosure relates to an agent, in particular a detergent or cleaning agent, which contains a lipase as defined herein. Furthermore, the present disclosure concerns a method for cleaning textiles as well as to the use of the agent as contemplated herein for removing stains.

### CROSS-REFERENCE TO RELATED APPLICATION

This application is a U.S. National-Stage entry under 35 U.S.C. § 371 based on International Application No. PCT/EP2018/064852, filed Jun. 6, 2018, which was published under PCT Article 21(2) and which claims priority to German Application No. 10 2017 209 869.4, filed Jun. 12, 2017, which are all hereby incorporated in their entirety by reference.

### TECHNICAL FIELD

The present disclosure relates to the field of enzyme technology, in particular the lipolytic action of enzymes on fats and oils as used in detergents or cleaning agents, for example. The present disclosure relates to an agent, in particular a detergent or cleaning agent, which contains a lipase as defined herein. Furthermore, the present disclosure relates to a method for cleaning textiles as well as to the use of the agent as contemplated herein for removing stains.

### BACKGROUND

Lipases belong to the most technically important enzymes of all. Their use in detergents and cleaning agents is well established in the industry and they are contained in practically all modern, high-performance detergents and cleaning agents. Lipases are enzymes which catalyse the hydrolysis of ester bonds in lipid substrates, in particular in fats and oils, and thus belong to the esterase group. Lipases are typically enzymes which can cleave a multitude of substrates, for example aliphatic, alicyclic, bicyclic and aromatic esters, thioesters and activated amines. Lipases are used for the removal of stains containing fats by catalysing their hydrolysis (lipolysis). Lipases with broad substrate spectra are in particular used where non-homogeneous raw materials or mixtures of substrates have to be reacted, i.e., for example, in detergents and cleaning agents, because stains can be formed from differently constituted fats and oils. The lipases employed in detergents or cleaning agents which are known in the prior art usually have microbial origins and as a rule derive from bacteria or fungi, for example of the genera Bacillus, Pseudomonas, Acinetobacter, Micrococcus, Humicola, Trichoderma or Trichosporon. Lipases are usually produced using suitable microorganisms and employing biotechnological methods which are known per se, for example via transgenic expression hosts from the genera Bacillus or via filamentous fungi.

As an example, European patent application EP 0 443 063 discloses a lipase intended for detergents and cleaning agents from Pseudomonas sp. ATCC 21808. Japanese patent application JP 1225490 discloses a lipase from Rhizopus oryzae. In general, only selected lipases are primarily suitable for use in liquid surfactant-containing preparations. In preparations of this type, many lipases do not have a sufficient catalytic power or stability, Particularly in washing processes, which are generally carried out at temperatures which are above about 20° C., many lipases exhibit thermal instability which in turn leads to unsatisfactory catalytic activity during the washing process. This problem is even more serious in phosphonate-containing liquid surfactant preparations, for example because of the complex-forming properties of phosphonates or because of disadvantageous interactions between the phosphonate and the lipase.

As a consequence, lipases and surfactant-containing liquid formulations from the prior art suffer from the disadvantage that they frequently do not exhibit a satisfactory lipolytic activity in the temperature ranges demanded for a washing process, and therefore do not exhibit optimal cleaning performance on lipase-sensitive stains.

### BRIEF SUMMARY

This disclosure provides an agent, such as a detergent or cleaning agent, that includes a lipase which has at least about 65% sequence identity with the amino acid sequence given in SEQ ID NO: 1 over its entire length.

This disclosure also provides a method for cleaning textiles, including the step of applying the aforementioned agent to the textiles.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, the present disclosure sets forth discoveries that a lipase from Microbulbifer thermotolerans, as described herein, is active under washing process conditions and exhibits good lipolytic properties. The sequence for the lipase identified herein does not have any significant sequence homologies with lipases used up to now in detergents or cleaning agents. Thus, it offers many possibilities for broadening the genetic versatility of commercially used lipases and possibly also of modifying the performance spectrum by employing mutagenesis.

Thus, in a first aspect, the present disclosure is directed towards an agent, in particular a detergent or cleaning agent, exemplified in that it contains a lipase which has at least about 65% sequence identity with the amino acid sequence given in SEQ ID NO: 1 over its entire length.

In a further aspect, the present disclosure is directed towards a method for cleaning textiles, exemplified in that an agent as contemplated herein is employed in at least one step of the method.

In yet another aspect, the present disclosure is further directed towards the use of an agent as described herein, preferably a detergent or cleaning agent, particularly preferably a liquid detergent, for removing (greasy) stains.

The lipases as contemplated herein exhibit enzymatic activity, which means that they are capable of hydrolyzing fats and oils, in particular in a detergent or cleaning agent. Thus, a lipase as contemplated herein is an enzyme which catalyzes the hydrolysis of ester bonds in lipid substrates and thus is capable of cleaving fats or oils. Furthermore, the lipase as contemplated herein is preferably a ripe (mature) lipase, i.e. the catalytically active molecule without signalling and/or pro-peptide(s). Unless stated otherwise, the sequences which are provided also refer to mature (processed) enzymes.

In various preferred embodiments of the present disclosure, the lipase is a lipase which has at least about 70% sequence identity with the amino acid sequence given in SEQ ID NO: 1 over its entire length. In further preferred embodiments, the lipase contained in the agent as contemplated herein comprises or essentially consists of the amino acid sequence given in SEQ ID NO: 1. In various embodiments of the present disclosure, the present disclosure also encompasses lipases which are derived from the amino acid sequence in accordance with SEQ ID NO: 1, for example by employing mutagenesis. In various other embodiments, the present disclosure also encompasses lipases which can be obtained by the expression of a nucleotide sequence which codes for a protein in accordance with SEQ ID NO: 1. In one aspect of the present disclosure, the present disclosure also encompasses nucleotide sequences which, with respect to the nucleotide sequence which codes for the protein in accordance with SEQ ID NO: 1, over its entire length has at least about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4% or about 99.6% identity therewith, with the proviso that the native sequence which codes for the lipase from Microbulbifer thermotolerans is excluded.

In various embodiments of the present disclosure, the lipase comprises an amino acid sequence which, with respect to the amino acid sequence given in SEQ ID NO: 1, over its entire length has at least about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4% or about 99.6% identity therewith or includes such a sequence.

In various other embodiments, the agent is exemplified in that

(a) the lipase is obtainable from a lipase as defined above as the starting molecule by one or more conservative amino acid substitutions; and/or
(b) the lipase is obtainable from a lipase as defined above as the starting molecule by fragmentation, deletion, insertion or substitution mutagenesis and comprises an amino acid sequence which, over a length of at least about 205, about 210, about 220, about 230, about 240, about 245, about 250, about 260, about 270, about 280, about 290, about 300, about 301, about 302, about 303, about 304, about 305, about 306, about 307, about 308, about 309, about 310, about 311, about 312, about 313, about 314 or about 315 consecutive amino acids, matches the starting molecule.

The agents as contemplated herein preferably contain the lipase in a quantity of from about 0.00001-about 1% by weight, more preferably in a quantity of from about 0.0001-about 0.5% by weight, particularly preferably in a quantity of from about 0.001-about 0.1% by weight, respectively with respect to the active protein.

The determination of the identity of nucleic acid or amino acid sequences is carried out by employing a sequence alignment. This sequence alignment is based on the BLAST algorithm which is established and in general use in the prior art (see, for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S. 3389-3402) and is based on the principle that similar sequences of nucleotides or amino acids in the nucleic acid or amino acid sequences are aligned with each other. A tabular correlation of the relevant positions is described as an alignment. A further algorithm which is available in the prior art is the FASTA algorithm. Sequence alignments (alignments), in particular multiple sequence alignments, are produced using computer programs. An example of a frequently used program is the Clustal series (see, for example Chenna et al. (2003): Multiple sequence alignment with the Clustal series of programs. Nucleic Acid Research 31, 3497-3500), T-Coffee (see, for example, Notredame et al. (2000): T-Coffee: A novel method for multiple sequence alignments. J. Mol. Biol. 302, 205-217) or programs which are based on these programs or algorithms. It is also possible to use sequence alignments using the computer program Vector NTI® Suite 10.3 (Invitrogen Corporation, 1600 Faraday Avenue, Carlsbad, Calif., USA) with the predetermined standard parameters; its AlignX module for sequence alignments is based on ClustalW.

An alignment of this type permits a statement to be made regarding the similarity of the aligned sequences to each other. It is usually expressed as the percentage identity, i.e. the proportion of identical nucleotides or amino acid residues in the same position or in a position corresponding to the alignment. The other term which is used, “homology”, as used for amino acid sequences, takes conserved amino acid exchanges into consideration, i.e. amino acids with similar chemical activity, because these usually have similar chemical activities within the protein. Thus, the similarity of the aligned sequences may also be given as the percentage homology or the percentage similarity. Identity and/or homology details may concern whole polypeptides or genes or just individual regions. Homology or identical regions of different nucleic acid or amino acid sequences are thus defined by agreements in the sequences. Such regions usually have identical functions. They may he small and only comprise a few nucleotides or amino acids. Frequently, such small regions carry out essential functions as regards the overall activity of the protein. It may therefore be of assistance to refer to sequence matches only for individual or, if appropriate, for small regions. Unless otherwise stated, the identity or homology details in the present application refer, however, to the total length of the respective nucleic acid or amino acid sequence which is given.

In various embodiments, the lipase comprises an amino acid sequence which, over its entire length, has at least about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4% or about 99.6% homology with the amino acid sequence given in SEQ ID NO: 1.

In a further embodiment of the present disclosure, the lipase has lipolytic activity compared with that of a lipase which comprises an amino acid sequence which corresponds to the amino acid sequences given in SEQ ID NO: 1, is not significantly reduced, i.e. has at least about 70%, about 75%, about 80%, about 85%, about 90%, about 95% of the reference activity. The lipolytic activity may be determined in a washing system which contains a detergent in a dose of between about 4.5 and about 7.0 grams per litre of washing liquor as well as the lipase, wherein the lipases to be compared are used at the same concentrations (with respect to active protein) and the lipolytic activity is determined as described herein. As an example, the washing process may be carried out for about 60 minutes at a temperature of about 60° C. and the water has a water hardness of between about 15.5° and about 16.5° (German hardness). The concentration of the lipase in the detergent specific for this washing system is from about 0.00001-about 1% by weight, preferably from about 0.0001-about 0.5% by weight, particularly preferably from about 0.001-about 0.1% by weight, with respect to active, purified protein.

A preferred liquid detergent for a washing system of this type is composed as follows (all details as a percentage by weight): anionic surfactant from about 5-about 7%, builder (for example citric acid and phosphonates) from about 0-about 1%, sodium hydroxide from about 0-about 1%, palm kernel oil fatty acid from about 0-about 1%, glycerine from about 0-about 1%, sodium chloride from about 1-about 3%, boric acid from about 0-about 1%, other additives (preservatives, defoaming agents, optical brighteners, colorant, fragrance) from about 0-1% and the remainder as demineralized water. Preferably, the dosage of the liquid detergent is between about 4.5 and about 6.0 grams per litre of washing liquor, for example about 4.7, about 4.9 or about 5.9 grams per litre of washing liquor. Preferably, washing is carried out in a pH range of between pH of about 8 and pH of about 10.5, preferably between pH of about 8 and pH of about 9.

In the context of the present disclosure, the determination of the lipolytic activity is carried out at about 40° C. using a liquid detergent as given above, wherein the washing process is preferably carried out for about 60 minutes.

The degree of whiteness, i.e. the lightening of the stains, as a measure of the cleaning performance is determined using optical measurement methods, preferably photometric methods. A suitable instrument in this regard is, for example, the Minolta CM508d spectrometer. Usually, the instruments used for the measurement are calibrated ahead of time with a white standard, preferably with a white standard which has been supplied.

Because lipases which have the same activity are used, this ensures that even in the case of a divergence in the ratios of the active substance to the total protein (the values for the specific activity), the respective enzymatic properties, i.e., for example, the cleaning performance with specific stains, may be compared. In general, a low specific activity can be balanced out by adding a larger quantity of protein.

The lipase activity may also be determined in a manner which is usual in the art, and in fact preferably as described by Bruno Stellmach, “Bestimmungsmethoden Enzyme für Pharmazie, Lebensmittelchemie, Technik, Biochemie, Biologie, Medizin” [Enzyme Determination Methods for Pharmacy, Food Chemistry, Technology, Biochemistry, Biology, Medicine] (Steinkopff Verlag Darmstadt, 1988, p. 172ff). In this method, lipase-containing samples are added to an olive oil emulsion in emulsifier-containing water and incubated at about 30° C. at a pH of about 9.0. This releases fatty acids. These are titrated using an autotitrator over about 20 minutes with continuous about 0.01 N sodium hydroxide so that the pH remains constant (pH-stat titration). The determination of the lipase activity is carried out with the aid of the sodium hydroxide consumption and with respect to a reference lipase sample.

An alternative test for establishing the lipolytic activity of the lipases as contemplated herein is an optical measurement method, preferably a photometric method. The test which is suitable in this case comprises the lipase-dependent cleavage of a para-nitrophenol butyrate substrate (pNP butyrate). This is cleaved by the lipase into para-nitrophenol and butyrate. The presence of para-nitrophenolate can be determined using a photometer, for example the Tecan Sunrise instrument, and XFLUOR software, at about 405 nm, and this allows the enzymatic activity of the lipase to be deduced.

Proteins can be categorized into groups of immunologically related proteins by employing the reaction with an antiserum or a specific antibody. The members of such a group have the same antigenic determinants which are recognized by an antibody. Thus, they resemble each other so closely structurally that they are recognized by an antiserum or specific antibodies. In a further aspect, then, the present disclosure provides lipases which have at least one and in increasing order of preference two, three or four antigenic determinants which match with a lipase used in an agent as contemplated herein. Because of their immunological matches with the lipases used as contemplated herein, such lipases are so similar in structure that it can be assumed that they have an identical function.

Further lipases used in the agents as contemplated herein may comprise further amino acid modifications compared to the lipase described in SEQ ID NO: 1, in particular amino acid substitutions, insertions or deletions. Lipases of this type are developed further, for example by targeted genetic modification, i.e. using mutagenesis methods, and optimized for specific purposes or as regards special properties (for example as regards their catalytic activity, stability, etc). Furthermore, nucleic acids which code for the lipases employed may be introduced in recombination strategies and therefore be used for the production of completely novel lipases or other polypeptides.

The aim is to introduce specific mutations such as substitutions, insertions or deletions into the known molecules in order, for example, to improve the cleaning performance of enzymes as contemplated herein. In this regard, the surface charges and/or the isoelectric point of the molecules in particular and thus their interactions with the substrate may be modified. Thus, for example, the net charge of the enzyme may be modified in order to influence the substrate binding in this manner, in particular for use in detergents and cleaning agents. Alternatively or as a supplement, by employing one or more relevant mutations, the stability of the lipase can be enhanced still further and in this manner, their cleaning performances can be improved. Advantageous properties of individual mutations, for example individual substitutions, may be supplemented. A lipase which has already been optimized as regards specific properties, for example as regards its activity, may thus be further developed in the context of the present disclosure.

Thus, in a further aspect, the present disclosure concerns an agent containing a lipase which can be obtained from a lipase as described above as the starting molecule via single or multiple conservative amino acid substitution. The term “conservative amino acid substitution” means the exchange (substitution) of one amino acid residue for another amino acid residue, wherein this exchange does not result in a modification of the polarity or charge at the position of the exchanged amino acid, for example the exchange of a non-polar amino acid residue for another non-polar amino acid residue. Examples of conservative amino acid substitutions in the context of the present disclosure are: G=A=S, I=V=L=M, D=E, N=Q, K=R, Y=F, S=T, G=A=I=V=L=M=Y=F=W=P=S=T. In this manner, the homology of lipases modified in this manner with the lipase with SEQ ID NO: 1 is advantageously as defined above.

As an alternative or as a supplement, the lipase can be obtained from a lipase contained in an agent as contemplated herein as a starting molecule by fragmentation, deletion, insertion or substitution mutagenesis and comprises an amino acid sequence which, over a length of at least about 205, about 210, about 220, about 230, about 240, about 245, about 250, about 260, about 270, about 280, about 290, about 300, about 301, about 302, about 303, about 304, about 305, about 306, about 307, about 308, about 309, about 310, about 311, about 312, about 313, about 314 or about 315 consecutive amino acids, matches the starting molecule.

Thus, for example, it is possible to delete individual amino acids at the ends or in the loops of the enzyme without losing or reducing the hydrolytic activity in this manner Furthermore, by employing fragmentation, deletion, insertion or substitution mutagenesis of this type, even the allergenicity of relevant enzymes can be reduced, for example, and thus their overall usefulness can be improved. Advantageously, the enzymes also retain their hydrolytic activity following mutagenesis, i.e. their hydrolytic activity corresponds to at least that of the starting enzyme, i.e. in a preferred embodiment, the hydrolytic activity is at least about 80%, preferably at least about 90% of the activity of the starting enzyme. Even further substitutions may exhibit advantageous effects. Both individual as well as multiple consecutive amino acids may be exchanged for other amino acids.

In various embodiments, in addition to the sequence given in SEQ ID NO: 1, the lipase may comprise one or more other N-terminal or C-terminal amino acids. In specific embodiments, such N-terminal peptides may be the naturally present signal peptides for the lipase, or may even be an individual methionine residue.

In one aspect, the present disclosure concerns an agent which contains a lipase as defined herein. Preferably, the agent is a detergent and cleaning agent.

Unless explicitly stated otherwise, all of the percentage details which are provided in connection with the compositions/agents described herein refer to the % by weight, respectively with respect to the respective mixture/the respective agent.

In the context of the present disclosure, “fatty acids” or “fatty alcohols” or their derivatives—unless stated otherwise—represent branched or unbranched carboxylic acids or alcohols or their derivatives, preferably containing about 6 to about 22 carbon atoms. In particular, oxo-alcohols or their derivatives which can be obtained using the ROELEN oxo synthesis, for example, may also be employed.

hereinafter, whenever an “alkaline earth metal” is mentioned as the counter-ion for monovalent anions, then this means that the alkaline earth metal is naturally present in only half the quantity as the anion, in order to balance the charges.

The subject matter of the present disclosure encompasses all envisageable types of detergents or cleaning agents, both concentrates and also agents which are to be used undiluted, for use on a commercial scale, in the washing machine or for hand washing. Thus, this includes, for example, detergents for textiles, carpets or natural fibres for which the description “detergent” is employed. Furthermore, in the context of the present disclosure, the term “detergent and cleaning agent” includes washing auxiliaries which are added when washing laundry manually or mechanically in order to obtain a further effect. Furthermore, in the context of the present disclosure, “detergents and cleaning agents” also includes textile pre-treatment and post-treatment agents, i.e. those agents with which the laundry to be washed is brought into contact before carrying out the washing proper, for example to loosen stubborn stains, and also those agents which provide the laundry with further desirable properties such as a pleasant feel, crease-resistance or reduced static electricity in a step which follows the actual textile washing step. Fabric softeners, inter alia, are included in these latter agents.

The detergents or cleaning agents as contemplated herein, which may be in the form of powdered solids, in post-compacted particles, as homogeneous solutions, gels or suspensions, may contain any known agents which are usual ingredients in agents of this type in addition to the lipase described above; preferably, at least one further ingredient is present in the agent. In particular, the agents as contemplated herein may contain surfactants, builders, bleaching agents, in particular peroxy compounds, or bleach activators. Furthermore, they may contain water-miscible organic solvents, other enzymes, sequestrating agents, electrolytes, pH regulators and/or other adjuvants such as optical brighteners, anti-redeposition agents, foam regulators as well as colorants and fragrances, as well as combinations thereof.

In particular, a combination of the agent as contemplated herein with one or more other ingredient(s) is advantageous, because in preferred embodiments as contemplated herein, an agent of this type has an improved cleaning performance because of synergistic effects which arise. In particular, a synergistic effect of this type may be obtained by employing a combination of the agent as contemplated herein with a surfactant and/or a builder and/or a peroxy compound and/or a bleach activator.

Advantageous ingredients for the agents as contemplated herein are disclosed in the international patent application WO 2009/121725, beginning on page 5, penultimate paragraph and ending after the second paragraph on page 13. Specific reference is made to this disclosure and the disclosure content therein is specifically incorporated into the present patent application.

These and other aspects, features and advantages of the present disclosure will become apparent to the person skilled in the art from a study of the following detailed description and claims. In this regard, every feature from one aspect of the present disclosure may be employed in any other aspect of the present disclosure. Furthermore, clearly, the examples contained herein are intended to describe and illustrate the present disclosure, but do not limit and in particular, the present disclosure is not limited to these examples. Unless stated otherwise, all percentages are given as the % by weight with respect to the total weight of the composition. Numerical ranges which are given in the format “from x to y” include the cited values. When a plurality of numerical ranges are given in this format, clearly, all ranges which arise by a combination of the various limits, are also included.

In addition to the lipase, the agents as contemplated herein preferably also contain at least one compound from the class formed by surfactants, in particular selected from anionic and non-ionic, and also from cationic, zwitterionic or amphoteric surfactants.

Examples of suitable anionic surfactants are surfactants with formula (I):

R—SO₃⁻Y⁺ (I).

In this formula (I), R represents a linear or branched, unsubstituted alkylaryl residue. Y represents a monovalent cation or the nth part of an n-valent cation; of these, alkali metal ions are preferred and of these, Na⁺ or K⁺ are preferred, wherein Na⁺ is most preferred. Further cations Y⁺ may be selected from NH₄⁺, ½Zn²⁺, ½Mg²⁺, ½Ca²⁺, ½Mn²⁺, and mixtures thereof.

“Alkylaryl”, as used herein, refers to an organic residue which includes an alkyl residue and an aromatic residue. Typical examples of residues of this type include but are not restricted to alkylbenzene residues such as benzyl, butylbenzene residues, nonylbenzene residues, decylbenzene residues, undecylbenzene residues, dodecylbenzene residues, tridecylbenzene residues and the like.

In various embodiments, surfactants of this type are selected from linear or branched alkylbenzene sulphonates with formula A-1:

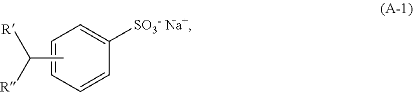

in which R′ and R″ together contain about 9 to about 19, preferably about 11 to about 15, and in particular about 11 to about 13 C atoms. A more particularly preferred representative may be described by the formula A-1a:

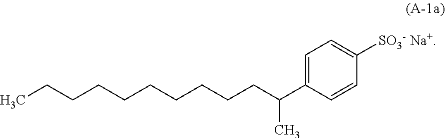

In various embodiments, the compound with formula (I) is preferably the sodium salt of a linear alkylbenzene sulphonate.

In agents as contemplated herein, the at least one compound from the class formed by anionic surfactants with formula (I) is contained in the detergent or cleaning agent in a quantity of from about 0.001 to about 30% by weight, preferably from about 0.001-about 10% by weight, more preferably from about 2-about 6% by weight, yet more preferably from about 3-about 5% by weight, respectively with respect to the total weight of the cleaning agent.

In various embodiments, the agents as contemplated herein preferably contain an anionic surfactant with formula:

R¹—O-(AO)ₙ—SO₃⁻X⁺ (II).

In this formula (II), R¹ represents a linear or branched, substituted or unsubstituted alkyl, aryl or alkylaryl residue, preferably a linear unsubstituted alkyl residue, particularly preferably a fatty alcohol residue. Preferred residues R¹ are selected from decyl, undecyl, dodecyl, tridecyl, tetradecyl, pentadecyl, hexadecyl, heptadecyl, octadecyl, nonadecyl, eicosyl residues and mixtures thereof, wherein the representatives with a linear arrangement of carbon atoms are preferred. Particularly preferred residues R¹ are derived from C₁₂-C₁₈-fatty alcohols, for example from coconut oil alcohol, tallow fatty alcohol, lauryl, myristyl, cetyl or stearyl alcohol or from C₁₀-C₂₀ oxoalcohols.

AO represents an ethylene oxide (EO) or propylene oxide (PO) group, preferably an ethylene oxide group. The index n represents a whole number from about 1 to about 50, preferably from about 1 to about 20 and in particular from about 2 to about 10. More particularly preferably, n represents the numbers 2, 3, 4, 5, 6, 7 or 8. X represents a monovalent cation or the nth part of an n-valent cation; of these, alkali metal ions are preferred and of these, Na⁺ or K⁺ are preferred, wherein Na⁺ is most preferred. Further cations X+ may be selected from NH₄⁺, ½Zn²⁺, ½Mg²⁺, ½Ca²⁺, ½Mn²⁺, and mixtures thereof.

In summary, then, in various embodiments, the agents contain at least one anionic surfactant selected from fatty alcohol ether sulphates with formula A2:

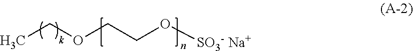

in which k=about 11 to about 19, n=2, 3, 4, 5, 6, 7 or 8. Particularly preferred representatives are Na—C₁₂₋₁₄ fatty alcohol ether sulphates containing 2 EO (k=about 11-about 13, n=2 in formula A-2).

In various embodiments, the cleaning agent contains the at least one anionic surfactant with formula (II) in a quantity of from about 2-about 10% by weight, preferably from about 3-about 8% by weight, with respect to the total weight of the cleaning agent.

Further anionic surfactants which may be used are alkyl sulphates with formula:

R²—O—SO₃⁻X⁺ (III).

In this formula (III), R² represents a linear or branched, substituted or unsubstituted alkyl residue, preferably a linear unsubstituted alkyl residue, particularly preferably a fatty alcohol residue. Preferred residues R² are selected from decyl, undecyl, dodecyl, tridecyl, tetradecyl, pentadecyl, hexadecyl, heptadecyl, octadecyl, nonadecyl, eicosyl residues and mixtures thereof, wherein the representatives with a linear arrangement of C atoms are preferred. Particularly preferred residues R² are derived from C₁₂-C₁₈-fatty alcohols, for example from coconut oil alcohol, tallow fatty alcohol, lauryl, myristyl, cetyl or stearyl alcohol or from C₁₀-C₂₀ oxoalcohols. Y represents a monovalent cation or the nth part of an n-valent cation; of these, alkali metal ions are preferred and of these, Na⁺ or K⁺ are preferred, wherein Na⁺ is most preferred. Further cations Y+ may be selected from NH₃⁺, ½Zn²⁺, ½Mg²⁺, ½Ca²⁺, ½Mn²⁺, and mixtures thereof.

In various embodiments, these surfactants are selected from fatty alcohol sulphates with formula A-3:

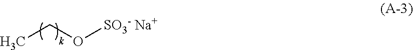

in which k=from about 11 to about 19. More particularly preferred representatives are Na—C₁₂₋₁₄ fatty alcohol sulphates (k=from about 11-13 in formula A-3).

In various embodiments, in addition to the anionic surfactants described above, in particular those with formulae (I)-(III), or as an alternative thereto, the agent may contain at least one other surfactant. Particular examples of alternative or additional surfactants that may be mentioned are further anionic surfactants, non-ionic surfactants and mixtures thereof, and also cationic, zwitterionic and amphoteric surfactants.

In various embodiments, the agents comprise at least one non-ionic surfactant, in particular at least one fatty alcohol alkoxylate.

Suitable non-ionic surfactants are those with formula:

R³—O-(AO)ₘ—H (IV),

in which
R³ represents a linear or branched, substituted or unsubstituted alkyl residue,
AO represents an ethylene oxide (EO) or propylene oxide (PO) group,
m represents whole numbers from about 1 to about 50.

In the aforementioned formula (IV), R³ represents a linear or branched, substituted or unsubstituted alkyl residue, preferably a linear unsubstituted alkyl residue, particularly preferably a fatty alcohol residue. Preferred residues R² are selected from decyl, undecyl, dodecyl, tridecyl, tetradecyl, pentadecyl, hexadecyl, heptadecyl, octadecyl, nonadecyl, eicosyl residues and mixtures thereof, wherein the representatives with a linear arrangement of C atoms are preferred. Particularly preferred residues R³ are derived from C₁₂-C₁₈ fatty alcohols, for example from coconut oil alcohol, tallow fatty alcohol, lauryl, myristyl, cetyl or stearyl alcohol or from C₁₀-C₂₀ oxoalcohols.

AO represents an ethylene oxide (EO) or propylene oxide (PO) group, preferably an ethylene oxide group. The index m represents a whole number from about 1 to about 50, preferably from about 1 to about 20 and in particular from about 2 to about 10. More particularly preferably, m represents the numbers 2, 3, 4, 5, 6, 7 or 8.

In summary, preferred fatty alcohol alkoxylates which are used are compounds with formula:

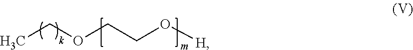

in which k=from about 11 to about 19, m =2, 3, 4, 5, 6, 7 or 8. More particularly preferred representatives are C₁₂₋₁₈ fatty alcohols containing 7 EO (k=about 11-about 17, m=7 in formula (V)).

Further non-ionic surfactants which may be contained in the agents described in the context of the present disclosure include but are not restricted to alkylglycosides, alkoxylated fatty acid alkyl esters, amine oxides, fatty acid alkanolamides, hydroxy mixed ethers, sorbitan fatty acid esters, polyhydroxy fatty acid amides and alkoxylated alcohols.

Examples of suitable amphoteric surfactants are betaines with formula (Rⁱⁱⁱ)(R^{iv})(R^{v})N⁺CH₂COO, in which Rⁱⁱⁱ represents an alkyl residue which may optionally be interrupted by heteroatoms or groups of heteroatoms containing from about 8 to about 25, preferably 10 to 21 carbon atoms, and R^{iv} as well as R^{v} represent identical or different alkyl residues containing 1 to 3 carbon atoms, in particular C₁₀-C₁₈ is alkyldimethyl carboxymethylbetaine and C₁₁-C₁₇ alkylamidopropyl dimethylcarboxymethylbetaine.

Suitable cationic surfactants include, inter alia, the quaternary ammonium compounds with formula (R^{vi})(R^{vii})(R^{viii})(R^{iv})N⁺X, in which R^{vi} to R^{ix} represent four identical or different, in particular two long- and two short-chain alkyl residues and X represents an anion, in particular a halide ion, for example didecyldimethylammonium chloride, alkylbenzyldidecylammonium chloride and mixtures thereof. Further suitable cationic surfactants are the quaternary surface-active compounds, in particular containing a sulphonium, phosphonium, iodonium or arsonium group, which are also known to be antimicrobial substances. By using quaternary surface-active compounds with an antimicrobial action, the agent can be equipped with an antimicrobial action or optionally, the antimicrobial action which is already present due to other ingredients may be improved.

In various embodiments, the total quantity of the surfactants, with respect to the weight of the agent, is from about 2% to about 30% by weight, preferably from about 5% to about 25% by weight, yet more preferably from about 10% to about 20% by weight, most preferably from about 14% to about 18% by weight, wherein the (linear) alkylbenzene sulphonates are present in a quantity of at most from about 0.001% to about 30% by weight, preferably from about 0.001-10% by weight, more preferably from about 2-6% by weight, yet more preferably from about 3-5% by weight, with respect to the weight of the agent.

In addition to the lipase, detergents or cleaning agents as contemplated herein may contain further enzymes. These may be hydrolytic enzymes or other enzymes in a concentration that is appropriate for the effectiveness of the agent. Thus, in one embodiment as contemplated herein, agents are provided which comprise one or more enzymes. Preferred enzymes which may be employed are all enzymes which may deploy a catalytic activity in the agents as contemplated herein, in particular a protease, amylase, cellulase, hemicellulase, mannanase, tannase, xylanase, xanthanase, xyloglucanase, β-glucosidase, pectinase, carrageenase, perhydrolase, oxidase, oxidoreductase, cutinase or other lipases, as well as mixtures thereof. The enzymes are advantageously respectively contained in the agent in a quantity of about 1×10⁻⁸ to about 5% by weight with respect to the active protein. In increasing order of preference, each enzyme is present in a quantity of about 1×10⁻⁷-about 3% by weight, from about 0.00001-about 1% by weight, from about 0.00005-about 0.5% by weight, from about 0.0001% to about 0.1% by weight and particularly preferably from about 0.0001% to about 0.05% by weight in agents as contemplated herein, with respect to the active protein. Particularly preferably, the enzymes exhibit synergistic cleaning performances in respect of specific stains or marks, i.e. the enzymes contained in the composition of the agent support each other as regards their cleaning performance. Synergistic effects may occur not simply between different enzymes, but also between one or more enzymes and further ingredients of the agent as contemplated herein.

The amylase(s) is/are preferably an α-amylase. The hemicellulase is preferably a β-glucanase, a pectinase, a pullulanase and/or a mannanase. The cellulase is preferably a cellulase mixture or a single-component cellulase, preferably or primarily an endoglucanase and/or a cellobiohydrolase. The oxidoreductase is preferably an oxidase, in particular a choline oxidase, or a perhydrolase.

The proteases used are preferably alkaline serine proteases. They act as non-specific endopeptidases, which means that they hydrolyse any acid amide bonds located in the interior of peptides or proteins and thus cause the degradation of protein-containing stains on the item to be cleaned. Their optimum pH is usually clearly in the alkaline range.

The protein concentration may be determined with the aid of known methods, for example the BCA method (bicinchoninic acid; 2,2′-bichinolyl-4,4′-dicarboxylic acid) or the biuret method. The determination of the active protein concentration in this regard is carried out by employing a titration of the active centres using a suitable irreversible inhibitor (for proteases, for example phenylmethylsulphonyl fluoride (PMSF)) and determination of the residual activity (see M. Bender et al., J. Am. Chem. Soc. 88, 24 (1966), pp 5890-5913).

In the cleaning agents described herein, the enzymes to be used may furthermore be manufactured together with accompanying substances, for example from fermentation. In liquid formulations, the enzymes are preferably employed as liquid enzyme formulation(s).

As a rule, the enzymes are not used in the form of the pure protein, but rather are provided in the form of stabilized preparations which are capable of being stored and transported. These pre-formulated preparations include solid preparations which, for example, are obtained by granulation, extrusion or freeze drying or, in particular in the case of liquid or gelled agents, solutions of the enzymes, advantageously as concentrated as possible, low in water and/or supplemented with stabilizers or further auxiliary agents.

Alternatively, the enzymes may be encapsulated both in the case of the solid and also in the case of the liquid form of administration, for example by spray drying or extrusion of the enzyme solution together with a preferably natural polymer or in the form of capsules, for example those in which the enzymes are enclosed in a solidified gel or in those of the core-shell type in which an enzyme-containing core is coated with a protective layer which is imprevious to water, air and/or chemicals. In the case of laminated layers, other additional substances, for example stabilizers, emulsifying agents, pigments, bleaching or colorants may be introduced. Capsules of this type are applied using methods which are known per se, for example by shaking or roller granulation or in fluidized bed processes. Advantageously, granulates of this type made, for example, by applying polymeric film-forming agents, are low in dust and stable upon storage due to the coating.

It is also possible to manufacture two or more enzymes together, so that a single granulate may comprise several enzyme activities.

In various embodiments, the agent as contemplated herein may comprise one or more enzyme stabilizers. Thus, the agent as contemplated herein may furthermore contain an enzyme stabilizer, for example selected from the group including sodium formate, sodium sulphate, lower aliphatic alcohols and boric acid, as well as their esters and salts. Naturally, two or more of these compounds may be also used in combination. The salts of the cited compounds may also be used in the form of hydrates such as sodium sulphate decahydrate, for example.

The term “lower aliphatic alcohols” as used herein includes monoalcohols, diols and higher alcohols containing up to 6 carbon atoms. In this regard, polyols, for example glycerine, (mono)ethylene glycol, (mono) propylene glycol or sorbitol may be mentioned from the group formed by lower aliphatic alcohols; the present disclosure is not limited thereto.

In addition to the at least one enzyme stabilizer selected from the aforementioned group, an agent as contemplated herein may also contain at least one further stabilizer. Stabilizers of this type are known in the prior art.

Reversible protease inhibitors protect the enzymes contained in a detergent or cleaning agent from proteolytic degradation, by reversibly inhibiting the enzymatic activity of the proteases contained in the agent. Benzamidine hydrochloride, boric acids or their salts or esters are frequently used as the reversible protease inhibitors, above all including derivatives with aromatic groups, for example ortho-, meta- or para-substituted phenyl boric acids, in particular 4-formylphenyl boric acid, or the salts or esters of the cited compounds. Even peptide aldehydes, i.e. oligopeptides with a reduced C terminus, in particular those containing from about 2 to about 50 monomers, are used for this purpose. Ovomucoid and leupeptin are peptide-type reversible protease inhibitors.

Further enzyme stabilizers are amino alcohols such as mono-, di- and tri-ethanol- and propanol-amine and mixtures thereof, aliphatic carboxylic acids up to C₁₂, such as, for example, succinic acid, other dicarboxylic acids or salts of the cited acids. Even end group-terminated fatty acid amide alkoxylates are suitable for this purpose. In addition, some organic acids used as builders additionally act to stabilize an enzyme. Even calcium and/or magnesium salts are used for this purpose; calcium acetate is an example.

Polyamide oligomers or polymeric compounds such as lignin, water-soluble copolymers or cellulose ethers, acrylic polymers and/or polyamides stabilize the enzyme preparation against physical influences or variations in pH, inter alia. Polyamine-N-oxide-containing polymers simultaneously act as enzyme stabilizers and as colour transfer inhibitors. Other polymeric stabilizers are linear polyoxyalkylenes. Alkyipolyglycosides may also stabilize the enzymatic components of the agent as contemplated herein and could advantageously additionally increase their performance. Crosslinked N-containing compounds advantageously carry out a dual function as soil release agents and as enzyme stabilizers. Hydrophobic, non-ionic polymers in particular stabilize any cellulase that might be present.

Reducing agents and antioxidants increase the stability of the enzymes against oxidative decomposition; in this regard, for example, sulphur-containing reducing agents are conventional, for example sodium sulphite and reducing sugar.

In one embodiment, the agents in accordance with the present disclosure are liquid and contain water as the principle solvent, i.e. they are aqueous agents. The water content of the aqueous agent as contemplated herein usually amounts to from about 15% to about 70% by weight, preferably from about 20% to about 60% by weight. In various embodiments, the water content is more than about 5% by weight, preferably more than about 15% by weight and particularly preferably more than about 50% by weight, respectively with respect to the total quantity of agent.

In addition, non-aqueous solvents may be added to the agent. Suitable non-aqueous solvents include monoalcohols or polyalcohols, alkanolamines or glycol ethers, as long as they are miscible with water in the given concentration range. Preferably, the solvents are selected from ethanol, n-propanol, i-propanol, butanols, glycol, propane diol, butane diol, methylpropane diol, glycerine, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, methoxytriglycol, ethoxytriglycol, butoxytriglycol, 1-butoxyethoxy-2-propanol, 3-methyl-3-methoxybutanol, propylene glycol t-butyl ether, di-n-octyl ether as well as mixtures of these solvents.

The one or more non-aqueous solvents is/are usually present in a quantity of from about 0.15% to about 10% by weight, preferably from about 15% to about 8% by weight with respect to the total composition.

In addition to the components mentioned above, the agents as contemplated herein may contain further ingredients which further improve the application technology and/or aesthetic properties of the cleaning agent. These include, for example, additives for improving the draining and drying behaviour, for adjusting the viscosity and/or for stabilization, as well as further adjuvants and additives which are normal in cleaning agents such as, for example, UV stabilizers, fragrances, pearlescent agents, colorants, corrosion inhibitors, preservatives, bitter substances, organic salts, disinfectants, structurizing polymers, defoaming agents, encapsulated ingredients (for example encapsulated fragrance), pH-adjusting agents as well as skin feel-improving or caring additives.

An agent as contemplated herein, in particular a detergent or cleaning agent, preferably contains at least one water-soluble and/or water-insoluble organic and/or inorganic builder.

In particular, builders which are in general use include ammocarboxylic acids and their salts, zeolites, silicates, carbonates, organic (co)builders and also phosphates—if there are no ecological impediments to using them. Preferably, however, the agents are phosphate-free.

Organic water-soluble builder substances includes polycarboxylic acids, in particular citric acid and saccharic acids, monomeric and polymeric aminopolycarboxylic acids, in particular methylglycine diacetic acid, Nitrilotriacetic acid and ethylendiamine tetraacetic acid, as well as polyaspartic acid, polyphosphonic acid, in particular aminotris(methylenephosphonic acid), ethylenediaminetetrakis(methylenephosphonic acid) and 1-hydroxyethane-1,1-diphosphonic acid, polymeric hydroxy compounds such as dextrin as well as polymeric (poly-)carboxylic acids, polymeric acrylic acids, methacrylic acids, maleic acids and mixed polymers thereof, which may also have polymerized into them small proportions of polymerizable substances without a carboxylic acid functionality. Suitable, albeit less preferred compounds in this class are copolymers of acrylic acid or methacrylic acid with vinyl ethers, such as vinylmethyl ethers, vinylesters, ethylene, propylene and styrene, in which the proportion of the acid is at least about 50% by weight. In particular, the organic builder substances may be used for the manufacture of liquid agents in the form of aqueous solutions, preferably in the form of from about 30% to about 50% by weight aqueous solutions. All of the cited acids are as a rule used in the form of their water-soluble salts, in particular their alkali salts.

If desired, organic builder substances may be used in quantities of up to about 40% by weight, in particular up to about 25% by weight and advantageously from about 1% by weight to about 8% by weight. Quantities close to the cited upper limit are preferably used in the form of pastes or liquid, in particular hydrated agents as contemplated herein. Laundry post-treatment agents such as, for example, fabric softeners, may also optionally be free from organic builders.

Water-soluble inorganic builder materials which may in particular be considered are alkali silicates and, if there is no objection to their use, also polyphosphates, preferably sodium triphosphate. Crystalline or amorphous alkali aluminosilicates, if desired, may be used as the water-insoluble, water-dispersible inorganic builder materials in particular in quantities of up to about 50% by weight, preferably not over about 40% by weight and in liquid agents in particular in quantities of from about 1% by weight to about 5% by weight. These include crystalline sodium aluminosilicates in detergent quality, in particular zeolite A, P and optionally X, which are preferred. Quantities close to the cited upper limits are preferably used in solid, particulate agents. Suitable aluminosilicates in particular do not have any particles with a grain size of more than about 30 μm and preferably include at least about 80% by weight of particles with a dimension of less than about 10 μm.

Suitable substitutes or partial substitutes for the cited aluminosilicate are crystalline alkali silicates, which may be present alone or as a mixture with amorphous silicates. The alkali silicates which may be used in the agents as contemplated herein as builders are preferably present in a molar ratio of alkali oxide to SiO₂ of less than about 0.95, in particular of from about 1:1.1 to about 1:12, and may be amorphous or crystalline. Preferred alkali silicates are sodium silicates, in particular amorphous sodium silicates with a molar ratio of Na₂:SO₂ of from about 1:2 to about 1:2.8. Preferred crystalline silicates which may be present alone or as a mixture with amorphous silicates are crystalline phyllosilicates with general formula Na₂SiₓO₂ₓ₊₁·y H₂O, in which x, what is known as the modulus, is a number from about 1.9 to about 4 and y is a number from 0 to about 20; preferred values for x are 2, 3 or 4. Preferred crystalline phyllosilicates are those in which x in the cited general formula takes the value 2 or 3. In particular, both beta and also delta sodium disilicates (Na₂Si₂O₅.y H₂O) are preferred. Even practically anhydrous crystalline alkali silicates with the above general formula produced from amorphous alkali silicates, in which x is a number from about 1.9 to about 2.1, may be used in the agents as contemplated herein. In a further preferred embodiment of the agent as contemplated herein, a crystalline sodium phyllosilicate is used with a modulus of from about 2 to about 3, such as that which can be produced from sand and soda. Crystalline sodium silicates with a modulus in the range from about 1.9 to about 3.5 are used in a further preferred embodiment of the agent as contemplated herein. In the case in which an alkali aluminosilicate, in particular zeolite, is present as an additional builder substance, the ratio by weight of aluminosilicate to silicate, respectively with respect to anhydrous active substances, is preferably from about 1:10 to about 10:1. In agents which contain both amorphous as well as crystalline alkali silicates, the ratio by weight of amorphous alkali silicate to crystalline alkali silicate is preferably from about 1:2 to about 2:1, and in particular from about 1:1 to about 2:1.

If desired, builder substances are preferably contained in the agents as contemplated herein in quantities of up to about 60% by weight, in particular from about 5% by weight to about 40% by weight. Particularly preferably, water-soluble builders are used in liquid formulations. Laundry post-treatment agents as contemplated herein, such as fabric softeners. are preferably free from inorganic builders.

Polymeric thickening agents in the context of the present disclosure are those polycarboxylates which act as polyelectrolytes to cause thickening, preferably homo- and copolymers of acrylic acid, in particular acrylic acid copolymers such as acrylic acid-methacrylic acid copolymers, and those which are polysaccharides, in particular heteropolysaccharides, as well as other usual thickening polymers.

Suitable polysaccharides or heteropolysaccharides are polysaccharide gums, for example gum Arabic, agar, alginate, carrageenan and its salts, guar, guarana, tragacanth, gellan, rhamsan, dextran or xanthan gum and their derivatives, for example propoxylated guar, as well as mixtures thereof. Other polysaccharide thickening agents, such as starches or cellulose derivatives, may be used as an alternative to polysaccharide gum, but preferably in addition thereto, for example starches from a wide variety of origins and starch derivatives, for example hydroxyethyl starches, starch phosphate esters or starch acetates, or carboxymethylcellulose or its sodium salt, methyl, ethyl, hydroxyethyl, hydroxypropyl, hydroxypropylmethyl or hydroxyethylmethyl cellulose, or cellulose acetate.

Examples of acrylic acid polymers which are suitable as polymeric thickening agents have a high molecular weight with a polyalkenyl polyether, in particular an allyl ether from saccharose, pentaerythritol or propylene, crosslinked homopolymers of acrylic acid (INCI name: carbomer), which are also described as carboxyvinyl polymers.

However, the following acrylic acid copolymers are particularly suitable as polymeric thickening agents: (i) copolymers from two or more monomers from the group formed by acrylic acid, methacrylic acid and monoesters, preferably formed with C₁₋₄ alkanols (INCI name: Acrylates Copolymer), which includes copolymers from methacrylic acid, butyl acrylate and methyl methacrylate (CAS 25035-69-2) or from butyl acrylate and methyl methacrylate (CAS 25852-37-3); (ii) crosslinked high molecular weight acrylic acid copolymers, which includes the copolymers of C₁₀₋₃₀ alkylacrylates crosslinked with an allyl ether of saccharose or of pentaerythritol with one or more monomers from the group formed by acrylic acid, methacrylic acid and their monoesters, preferably formed with C₁₋₄ alkanols (INCI name: Acrylates/C10-30 Alkyl Acrylate Crosspolymer).

The quantity of polymeric thickening agent usually amounts to not more than about 8% by weight, preferably between about 0.1% and about 7% by weight, particularly preferably between about 0.5% and about 6% by weight, in particular between about 1% and about 5% by weight and most preferably between about 1.5% and about 4% by weight, for example between about 2% and about 2.5% by weight, with respect to the total weight of the agent.

In order to stabilize the agent as contemplated herein, in particular when the surfactant content is high, one or more dicarboxylic acids and/or their salts may be added, in particular a composition formed from Na salts of adipine, succinic acid and giutaric acid which is available, for example, under the trade name Sokalan® DSC. It is advantageously used in quantities of from about 0.1% to about 8% by weight, preferably from about 0.5% to about 7% by weight, in particular from about 1.3% to about 6% by weight and particularly preferably from about 2% to about 4% by weight, with respect to the total weight of the cleaning agent.

However, their use may be dispensed with, and so the agent as contemplated herein is preferably free from dicarboxylic acids (salts).

The detergents as contemplated herein may be compared with reference detergents in order to determine the enhanced anti-pilling performance of the agents as contemplated herein. A washing system of this type may be composed as follows (all details as a percentage by weight): reference agent: anionic surfactant from about 5-about 7%, builder (for example citric acid and phosphonates) from about 0-about 1%, sodium hydroxide from about 0-about 1%, palm kernel oil fatty acid from about 0-about 1%, glycerine from about 0-about 1%, sodium chloride from about 1-about 3%, boric acid from about 0-about 1%, other additives (preservatives, defoaming agents, optical brighteners, colorant, fragrance) from about 0-about 1% and the remainder as demineralized water. Agent as contemplated herein: anionic surfactant from about 5-about 7%, builder (for example citric acid and phosphonates) from about 0-about 1%, sodium hydroxide from about 0-about 1%, palm kernel oil fatty acid from about 0-about 1%, glycerine from about 0-about 1%, sodium chloride from about 1-about 3%, boric acid from about 0-about 1%, other additives (preservatives, defoaming agents, optical brighteners, colorant, fragrance) 0-about 1%, lipase as contemplated herein from about 0.001-about 0.1% and the remainder as demineralized water. Preferably, the dosage of the liquid detergent is between about 4.5 and about 6.0 grams per litre of washing liquor, for example 4.7, 4.9 or 5.9 grams per litre of washing liquor. Preferably, washing is carried out in a pH range of between pH of about 8 and pH of about 10.5, preferably between pH of about 8 and pH of about 9.

The embodiments of the present disclosure defined above comprise all solid, powdered, liquid, gelled or paste forms of administration of agents as contemplated herein which may optionally include a plurality of phases and may also be in a compressed or non-compressed form. The agent may be present as a free-flowing powder, in particular with a bulk density of from about 300 g/L to about 1200 g/L, in particular from about 500 g/L to about 900 g/L or from about 600 g/L to about 850 g/L. Furthermore, the solid forms of delivery of the agent include extrudates, granulates, tablets or pouches. Alternatively, the agent may also be liquid, gelled or a paste, for example in the form of a non-aqueous liquid detergent or a non-aqueous paste or in the form of an aqueous liquid detergent or a hydrated paste. Furthermore, the agent may be a single component system. Agents of this type include one phase. Alternatively, an agent may also include a plurality of phases. Thus, an agent of this type is divided into several components (multi-component system).

A further objective of the present disclosure is constituted by a method for cleaning textiles wherein an agent as contemplated herein is used in at least one step of the method.

In various embodiments, the method described above is exemplified in that the agent as contemplated herein is used at a temperature of from about 0-about 100° C., preferably from about 0-about 80° C., more preferably from about 30-about 70° C. and most preferably at from about 40-about 60° C.

This encompasses both manual and also mechanical methods, wherein mechanical methods are preferred. Methods for cleaning textiles are generally exemplified in that in a plurality of steps of the method, different substances with cleaning activity are applied to the item to be cleaned and is washed out after being allowed to work, or in that the item to be cleaned is treated in some way with a detergent or a solution or dilution of this agent. Any envisageable washing or cleaning method may be used in at least one of the steps of the method in order to enhance the use of the detergent or cleaning agent as contemplated herein; these therefore constitute embodiments of the present disclosure. All circumstances, objectives and embodiments which are described for the agents as contemplated herein are also applicable to this subject matter of the present disclosure. Thus, here, we shall direct attention expressly to the disclosure of the relevant section, with the indication that this disclosure is also valid for the present method as contemplated herein.

Because enzymes naturally already have a catalytic activity and these also occur in media which otherwise have no cleaning power such as, for example, in pure buffers, then an individual and/or the single step of such a method may include bringing a lipase into contact with the stain as the single cleaning active component, preferably in a buffer solution or in water. This constitutes a further embodiment of this disclosure.

Alternative embodiments of this disclosure are also constituted by methods for the treatment of textile raw materials or for the care of textiles, in which in at least one step of the method, an agent as contemplated herein is active. In this regard, methods for raw textile materials, fibres or textiles with synthetic components are preferred.

Furthermore, the present disclosure also encompasses the use of the agent described herein, for example as detergents or cleaning agents as described above, for the (improved) removal of stains, for example from textiles.

All circumstances, objectives and embodiments which have been described for the agent as contemplated herein and the lipase, are also applicable to the further subject matter of the present disclosure. Thus, here, we shall direct attention expressly to the disclosure of the relevant section, with the indication that this disclosure is also valid for the present method as contemplated herein, and for the uses as contemplated herein.

### EXAMPLES

### Example 1: Cloning and Expression

In an activity-based screening, five metagenome databases were screened. In this manner, 21 clones could be isolated, which exhibited activity on LB agar plates supplemented with tributyrin. Plasmid DNA could be isolated from 13 possible positive clones. The approximate insertion size was determined by employing enzymatic digestion. The isolated plasmid DNA was sequenced in steps and provided 11 different Open Reading Frames (ORF). One of the ORFs could be assigned to a lipase from Microbulbifer thermotolerans (MtLip).

For the purposes of recombinant expression of the target lipase MtLip, the gene was cloned in a pQE30 expression plasmid and transformed in the expression strain E. coli BL21 (DE3) pLys. 1 L of culture was drawn off. Induction was triggered with 0.1 mM IPTG and culturing was carried out at 37° C. for 24 h. Next, the cells were lysed using ultrasound. The raw extract obtained was used for a mini-washing test.

### Example 2: Washing Test

A washing test was carried out with the E. coli raw extract, in which the described lipase was expressed.

Washing was carried out in a conventional liquid detergent without enzymes (Table 1) at 40° C. in 16° dH Wasser for 1 h. The enzyme concentration was 0.32 μg lipase/millilitre of detergent solution, corresponding to a normal lipase concentration in detergents.

TABLE 1
Detergent matrix used A commercially available detergent
matrix (without optical brighteners, fragrance and
colorants) was used for the washing test:

| | % by weight of active |
|---|---|
| Chemical name | substance in the formulation |
| Anionic surfactant | 5-7% |
| Non-ionic surfactant | 3-5% |
| Builder (citric acid and phosphonates) | 0-1% |
| Sodium hydroxide | 0-1% |
| Palm kernel oil fatty acid | 0-1% |
| Glycerine | 0-1% |
| Sodium chloride | 1-3% |
| Boric acid | 0-1% |
| Propylene glycol laurate | — |
| Further additives (preservatives, | 0-1% |
| defoaming agents, optical brighteners, | |
| colorant, fragrance) | |
| Water | remainder |

Dose: 4.7 g/L

The following stains were used in the test:

| | | |
|---|---|---|
| 1. wfk 20D | Pigment/tallow | Polyester/cotton |
| 2. CS 61 | Beef fat, stained | Cotton |
| 3. CS 46b | Spent deep-frying fat | Cotton |
| | with violet colorant | |

The individual fabrics were punched out (diameter=10 mm) and placed in a microtitre plate. The detergent solution, with a final concentration of 4.0 g/L, was pre-heated. to 40° C. Next, the detergent solution and the enzyme were placed on the stain and incubated for 1 h at 40° C. and at 600 rpm. After washing, the stains were rinsed several times with clean water, dried and the brightness was determined using a colour measurement device.

The lighter the fabric, the better was the cleaning performance. Here, the L value was measured=lightness, and so the higher this was, the brighter it was. The brightness values for the test batches are recorded in Table 2.

Sample 1: Detergent without lipase

Sample 2: Detergent with MtLip

TABLE 2
Results

| | Sample 1 | Sample 2 |
|---|---|---|
| wfk 20D | 69.19 | 77.15 |
| CS 61 | 74.50 | 79.70 |
| CS 46b | 74.94 | 81.12 |

It is clear that the lipase as contemplated herein performed well on all three stains. Just one unit is considered to be a significant improvement in performance; here, an improvement of up to 7.9 units was obtained.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An agent, comprising a lipase which has at least about 65% sequence identity with the amino acid sequence given in SEQ ID NO: 1 over its entire length.

2. The agent as claimed in claim 1, wherein the lipase comprises an amino acid sequence which, with respect to the amino acid sequence given in SEQ ID NO: 1, over its entire length has at least about 66% identity therewith.

3. The agent as claimed in claim 1, wherein
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 205 consecutive amino acids, matches the starting molecule.

4. The agent as claimed in claim 1, wherein the lipase is included in the agent in a quantity of from about 0.00001-about 1% by weight.

5. The agent as claimed in claim 1, wherein
a. the agent comprises at least one additional ingredient selected from the group of surfactants, builders, bleaching agents, bleach activators, water-miscible organic solvents, further enzymes, sequestrating agents, electrolytes, pH regulators, optical brighteners, anti-redeposition agents, foam regulators, colorants and fragrances, as well as combinations thereof; and/or
b. the agent is in a solid or liquid form.

6. A method for cleaning textiles, comprising the step of applying an agent as claimed in claim 1 to the textiles.

7. (canceled)

8. The agent as claimed in claim 1, wherein the lipase comprises an amino acid sequence which, with respect to the amino acid sequence given in SEQ ID NO: 1, over its entire length has at least about 90% identity therewith.

9. The agent as claimed in claim 1, wherein the lipase comprises an amino acid sequence which, with respect to the amino acid sequence given in SEQ ID NO: 1, over its entire length has at least about 99.6% identity therewith.

10. The agent as claimed in claim 1, wherein
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule.

11. The agent as claimed in claim 2, wherein
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule.

12. The agent as claimed in claim 8, wherein
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule.

13. The agent as claimed in claim 9, wherein
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule.

14. The agent as claimed in claim 2, wherein the lipase is included the agent in a quantity of from about 0.001-about 0.1% by weight.

15. The agent as claimed in claim 8, wherein the lipase is included in the agent in a quantity of from about 0.001-about 0.1% by weight.

16. The agent as claimed in claim 9, wherein the lipase is included in the agent in a quantity of from about 0.001-about 0.1% by weight.

17. The agent as claimed in claim 1, wherein
a. the agent comprises at least one additional ingredient selected from the group of surfactants, builders, bleaching agents, bleach activators, water-miscible organic solvents, further enzymes, sequestrating agents, electrolytes, pH regulators, optical brighteners, anti-redeposition agents, foam regulators, colorants and fragrances, as well as combinations thereof; and
b. the agent is in liquid form.

18. The agent as claimed in claim 8, wherein
a. the agent comprises at least one additional ingredient selected from the group of surfactants, builders, bleaching agents, bleach activators, water-miscible organic solvents, further enzymes, sequestrating agents, electrolytes, pH regulators, optical brighteners, anti-redeposition agents, foam regulators, colorants and fragrances, as well as combinations thereof; and
b. the agent is in liquid form.

19. The agent as claimed in claim 1, wherein:
the lipase comprises an amino acid sequence which, with respect to the amino acid sequence given in SEQ ID NO: 1, over its entire length has at least about 90% identity therewith,
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule,
the lipase is included in the agent in a quantity of from about 0.001-about 0.1% by weight,
the agent comprises at least one additional ingredient selected from the group of surfactants, builders, bleaching agents, bleach activators, water-miscible organic solvents, further enzymes, sequestrating agents, electrolytes, pH regulators, optical brighteners, anti-redeposition agents, foam regulators, colorants and fragrances, as well as combinations thereof, and the agent is in liquid form.

20. The agent as claimed in claim 1, wherein:
the lipase comprises an amino acid sequence which, with respect to the amino acid sequence given in SEQ ID NO. 1, over its entire length has at least about 99.6% identity therewith,
(1) the lipase is obtained from a lipase as claimed in claim 1 as the starting molecule by one or more conservative amino acid substitutions; and/or
(2) the lipase is obtained from a lipase as claimed in claim 1 as the starting molecule by fragmentation, deletion, insertion or substitution mutagenesis and comprises an amino acid sequence which, over a length of at least about 315 consecutive amino acids, matches the starting molecule,
the lipase is included in the agent in a quantity of from about 0.001-about 0.1% by weight,
the agent comprises at least one additional ingredient selected from the group of surfactants, builders, bleaching agents, bleach activators, water-miscible organic solvents, further enzymes, sequestrating agents, electrolytes, pH regulators, optical brighteners, anti-redeposition agents, foam regulators, colorants and fragrances, as well as combinations thereof, and
the agent is in liquid form.
